(19) United States
(12) Patent Application Publication
(10) Pub. No.: US 2024/0366577 A1
(43) Pub. Date: Nov. 7, 2024

(54) CONTROLLED LONG-TERM DRUG DELIVERY AND METHODS THEREOF

(71) Applicant: THE JOHNS HOPKINS UNIVERSITY, Baltimore, MD (US)

(72) Inventors: Joshua DOLOFF, Towson, MD (US); Victoria LAI, Silver Spring, MD (US); Sarah Yoseph NESHAT, Baltimore, MD (US); Elia DUH, Lutherville, MD (US); James Joseph PITINGOLO, Berkeley Heights, NJ (US); Amanda Isabella RAKOSKI, Baltimore, MD (US); Lingli ZHOU, Baltimore, MD (US); Haining LU, Baltimore, MD (US)

(21) Appl. No.: 18/688,195

(22) PCT Filed: Sep. 2, 2022
(86) PCT No.: PCT/US2022/042480

§ 371 Date: Feb. 29, 2024

Related U.S. Application Data
(60) Provisional application No. 63/240,509, filed on Sep. 3, 2021.

(51) Int. Cl. A61K 31/44

(52) U.S. Cl. CPC A61K 31/44

(57) ABSTRACT

An anti-angiogenic composition is disclosed. The anti-angiogenic composition includes a multi-tyrosine kinase inhibitor having a chemical structure:

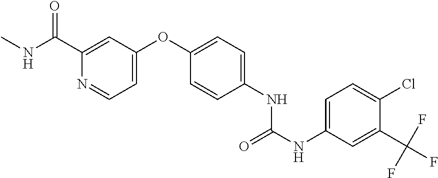 [SMILES: CNC(=O)c1cc(Oc2ccc(NC(=O)Nc3ccc(Cl)c(C(F)(F)F)c3)cc2)ccn1]

where the tyrosine kinase inhibitor has a crystalline structure having a single crystal polymorph. The anti-angiogenic composition including the tyrosine kinase inhibitor, as characterized by powder x-ray diffraction, has diffraction peaks at 2θ of 11.37 150 18.04 242 288 and 24.72. The tyrosine kinase inhibitor may have a particle size of from about 50 to about 300 microns. The tyrosine kinase inhibitor is injectable. The tyrosine kinase inhibitor has an anti-angiogenic efficacy as indicated by a reduction in endothelial cell marker cd31, vascular endothelial-cadherin (VE-CDH), smooth muscle pericyte marker alpha smooth muscle actin (ASMACT), or a combination thereof, as compared to a control composition.

### CROSS REFERENCE TO RELATED APPLICATIONS

This application is the national stage entry of International Patent Application No. PCT/US2022/042480, filed on Sep. 2, 2022, and published as WO 2023/034581 A1 on Mar. 9, 2023, which claims the benefit of U.S. Provisional Patent Application Ser. No. 63/240,509, filed on Sep. 3, 2021, which are hereby incorporated by reference herein in their entireties.

### TECHNICAL FIELD

The present teachings relate generally to long-term drug delivery and, more particularly, to crystallization methods for the synthesis of compositions for controlled long-term drug delivery.

### BACKGROUND

A significant challenge in the development and compositions of cancer medicine is achieving the safe and effective delivery of drugs to the right tissue at the right time. Despite being targeted, many drugs often result in side effects and lack of safety in patients following the common practice of global dissemination. Certain formulations of hydrophobic drug crystals, may show potential for reducing side effects, improving safety and efficacy, and provide controlled long-term release from a single dosage administration.

Since aberrant angiogenesis and tumor growth interfere with cancer cell elimination and contribute to metastatic progression, ultimately leading to poor patient outcomes, certain multi-tyrosine kinase inhibitor and multi-modal (anti-angiogenic and tumor cell cytotoxic) agents, may prove effective in sustained delivery and treatment of orthotopic triple negative breast cancer (TNBC) mouse model (4T1 in syngeneic BALB/c mice), anti-tumor activity. The engineering of crystalline drug delivery systems has implications in the treatment of cancer or other diseases where high enough constitutive drug levels are needed to maintain target saturation and inhibition while also preventing emergence of drug resistance, often seen with suboptimal dosing. Furthermore, the use of a single local, subcutaneous injection of a crystalline drug may be capable of improving specificity to reduce off-target effects. However, the formulation of drug crystals, from hydrophobic and otherwise difficult to solubilize small molecule chemical compounds, capable of providing constant drug release for weeks following a single injection have proven challenging.

The development of effective drug formulations capable of improving specificity and reducing off-target effects, which are also capable of providing constant drug release for weeks following a single injection of significant interest. Thus, drug formulations having safe, predictable, and targeted delivery of an anti-angiogenic and tumor cell cytotoxic agent are desirable. The development and compositions of cancer medicine is achieving the safe and effective delivery of drugs to the right tissue at the right time without significant side effects associated with global dissemination. Certain formulations of hydrophobic drug crystals may show potential for reducing side effects, improving safety and efficacy, and provide controlled long-term release from a single dosage administration. Furthermore, enabling the elimination of dangerous organic solvents in a drug formulation to deliver a hydrophobic drug would be advantageous. Certain crystalline drug compositions may allow one to obtain a pure, densely packed drug delivery depot or system, capable of tunable release kinetics that can last up to months or years long timescales, or alternatively be designed for shorter days to weeks times as well.

### Summary

The following presents a simplified summary in order to provide a basic understanding of some aspects of one or more embodiments of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later.

An anti-angiogenic composition is disclosed. The anti-angiogenic composition includes a multi-tyrosine kinase inhibitor having a chemical structure:

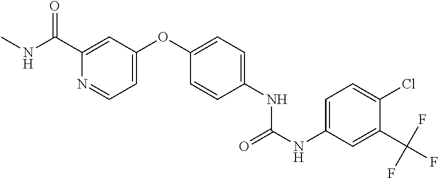 [SMILES: CNC(=O)c1cc(Oc2ccc(NC(=O)Nc3ccc(Cl)c(C(F)(F)F)c3)cc2)ccn1]

The anti-angiogenic composition includes where the tyrosine kinase inhibitor has a crystalline structure having a single crystal polymorph. The anti-angiogenic composition including the tyrosine kinase inhibitor, as characterized by powder x-ray diffraction, has diffraction peaks at 2θ of 11.37 150 18.04 242 288 and 24.72. The tyrosine kinase inhibitor may have a particle size of from about 5 to about 300 microns. The tyrosine kinase inhibitor may have a particle size of from about 150 to about 200 microns. The tyrosine kinase inhibitor may be crystallized using a solvent/anti-solvent crystallization reaction. The solvent may be ethyl acetate or alternatively, hexane. The tyrosine kinase inhibitor may be sonicated. The tyrosine kinase inhibitor is injectable. The tyrosine kinase inhibitor may include no carrier. The tyrosine kinase inhibitor is hydrophobic. The tyrosine kinase inhibitor has an anti-angiogenic efficacy as indicated by a reduction in endothelial cell marker cd31, vascular endothelial-cadherin (VE-CDH), smooth muscle pericyte marker alpha smooth muscle actin (ASMACT), or a combination thereof, as compared to a control composition

A multi-tyrosine kinase inhibitor composition is also disclosed. The multi-tyrosine kinase inhibitor composition also includes a crystalline sorafenib. The multi-tyrosine kinase inhibitor composition also includes where the crystalline sorafenib, as characterized by powder x-ray diffraction, has diffraction peaks at 2θ of 11.37, 12.50, 18.04, 22.42, 22.88, and 24.72. The multi-tyrosine kinase inhibitor composition may include where the crystalline sorafenib has a particle size of from about 5 to about 300 microns. The crystalline sorafenib may alternatively have a particle size of from about 150 to about 200 microns. The crystalline sorafenib may be injectable. The multi-tyrosine kinase inhibitor composition may include no carrier. The multi-tyrosine kinase inhibitor composition may be hydrophobic. The multi-tyrosine kinase inhibitor composition may be crystallized using a solvent/anti-solvent crystallization reaction, where the solvent is ethyl acetate and the anti-solvent is hexane.

An injectable drug composition is also disclosed. The injectable drug composition also includes a multi-tyrosine kinase inhibitor which may further include a compound of structural formula:

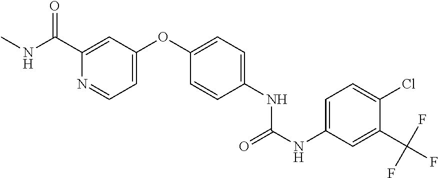 [SMILES: CNC(=O)c1cc(Oc2ccc(NC(=O)Nc3ccc(Cl)c(C(F)(F)F)c3)cc2)ccn1]

The composition may also include where the multi-tyrosine kinase inhibitor having a crystalline structure which may include a single crystal polymorph, the multi-tyrosine kinase inhibitor, as characterized by powder x-ray diffraction, has diffraction peaks at 2θ of 11.37, 12.50, 18.04, 22.42, 22.88, and 24.72, and the multi-tyrosine kinase inhibitor has a particle size of from about 150 to about 200 microns.

### BRIEF DESCRIPTION OF THE DRAWINGS

The accompanying drawings, which are incorporated in and constitute a part of this specification, illustrate embodiments of the present teachings and together with the description, serve to explain the principles of the disclosure. In the figures:

FIGS. 1A-1G depict several views and analyses related to development and characterization of sorafenib crystals, according to an embodiment.

FIGS. 2A-2E depicts various solution stability and tunable release metrics and data related to sorafenib crystals, according to an embodiment.

FIGS. 3A-3F are a series of methods and data related to evaluation of anti-tumor effects associated with single delivery of crystalline sorafenib, according to an embodiment.

FIGS. 4A-4E depict a series of images and data illustrating anti-angiogenic efficacy of sorafenib crystals, according to an embodiment.

FIG. 5 depicts a timeline of an experimental procedure, in accordance with the present disclosure.

FIGS. 6A-6D depict charts of fluorescent intensity as measured with the use of fundus fluorescein angiography (FFA), according to the present disclosure.

It should be noted that some details of the figures have been simplified and are drawn to facilitate understanding of the present teachings rather than to maintain strict structural accuracy, detail, and scale.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same, similar, or like parts.

The development and compositions of cancer medicine is achieving the safe and effective delivery of drugs to the right tissue at the right time without significant side effects associated with global dissemination. Certain formulations of hydrophobic drug crystals may show potential for reducing side effects, improving safety and efficacy, and provide controlled long-term release from a single dosage administration. Furthermore, the enablement of the elimination of dangerous organic solvents in a drug formulation to deliver an incredibly hydrophobic drug would be advantageous. Certain crystalline drug compositions may allow one to obtain a pure, densely packed drug delivery depot or system, capable of tunable release kinetics that can last as long as months or years long timescales, or alternatively be designed for shorter days to weeks times as well.

As described herein, new and effective drug formulations capable of improving specificity and reducing off-target effects, are described, capable of providing constant drug release for weeks following a single injection. The multi-tyrosine kinase inhibitor and multi-modal (anti-angiogenic and tumor cell cytotoxic) agent sorafenib, has been used herein, as aberrant angiogenesis and tumor growth interfere with cancer cell elimination and contribute to metastatic progression, ultimately responsible for poor patient outcomes. In certain embodiments as described herein, the crystal size (surface area:volume ratios) was tuned, imaged by SEM, and the controllability of drug delivery kinetics in in vitro drug release assays is demonstrated. Single and powder crystal X-ray diffraction (XRD) indicates that all the sorafenib crystals produced were the same polymorph and drug form. Furthermore, when utilized against an orthotopic triple negative breast cancer (TNBC) mouse model (4T1 in syngeneic BALB/c mice), anti-tumor activity from a single local, subcutaneous injection of crystalline sorafenib was established. This and other embodiments validate the advantages of engineering crystalline drug delivery systems and in the treatment of cancer or other diseases where high enough constitutive drug levels are needed to maintain target saturation and inhibition while also preventing emergence of drug resistance, often seen with suboptimal dosing.

In cancer, an extensive vasculature network is required to provide nutrients for rapid tumor development. A discrete step in tumorigenesis, the “angiogenic switch” is an analogy used to describe the balance between pro-and anti-angiogenic factors in the tumor environment for the induction of neovascularization. In normal vasculo/angiogenesis, the “switch” in adults is tightly regulated and largely turned “off”, except in instances such as wound healing, where vessels mature rapidly and readily become stable. By contrast, during tumorigenesis, the switch is much more dynamic. Stimuli such as oncogene activation or hypoxia from increased tissue mass will enable elevated pro-angiogenic factors, readily turning the switch “on” and promoting formation of immature vasculature to sustain ever-growing tumors with nutrients.

One common tactic in treating chronic angiogenesis, a common hallmark in cancer, is the inhibition of the vascular endothelial growth factor (VEGF)/receptor (VEGFR) as well as platelet derived growth factor receptor (PDGFR) pathways, which are required for vascularization and new vessel growth as well as maintenance of mature vasculature. One focus in oncology is the investigation of potential treatments that combine medications that pair various treatment mechanisms to overcome various aspects of cancer and are less prone to tumor cell resistance. Targeted therapies exist in the form of receptor inhibitors. Once such agent, sorafenib, was originally developed by BAYER to possess antitumor and anti-angiogenic efficacy by inhibiting multiple receptor tyrosine kinases (RTKs). Of note, sorafenib is an RTK inhibitor that targets a number of oncogenic-adjacent pathways, including CRAF & BRAF—both intracellular kinases that are key regulators of the ERK MAP kinase pathway for proliferation, differentiation, and migration; VEGFR-2, VEGFR-3, and PDGFR-b—required for the regulation of angiogenesis; and c-Kit and FLT-3—genes that regulates proliferation and differentiation of hematopoietic stem cells. Ultimately, however, efficacy of most single pathway inhibitors has been far from perfect. Aside from mutations resulting in receptor upregulation, these therapies are also subject to “on-target/off-tumor” effects—that is, they may not inhibit sites of tumor growth alone, and thus can potentially interfere with normal cell function. As such, an ideal drug delivery system would be one that allows a multi-targeted agent to be locally dosed at a sustained rate of release within an optimal target dose window. Such a drug delivery system could reduce side effects or emergence of cancer cell drug resistance. Research is currently being done on devices such as micropumps, biomaterial release systems, reservoirs, and the like. One delivery system of interest is the use of monolithic crystalline medications. Due to the purity, high density of drug per volume, as well as the ability to control rate of dissolution through morphology associated with monolithic crystalline medications, they may make an ideal candidate for local delivery to a tumor microenvironment.

Crystallization is an essential process for the formation of many chemical compounds during both the separation and purification steps in mass production. The natural formation of a crystal lattice is often reproducible due to the fact that the constituent atoms or molecules are typically discriminating, allowing only for the arrangement and attachment of related units. As such, resulting crystals are highly pure, a characteristic that is crucial for industries such as food and pharmaceutical, where mitigating risk of contamination is a primary objective. However, the challenge then becomes being able to synthesize crystals with consistent morphology—that is, crystal structure, shape, as well as uniform particle size distribution (PSD)—all of which are characteristics that affect functionality. In the pharmaceutical industry, crystallization is often only the first step in synthesis, which is then followed with a number of refinement stages to separate, dry, grind, and filter the product as a powder that can either be pressed into a tablet, suspended, encapsulated, or simply packaged as the powder itself. It is rare for the crystal itself to be sent to market for final use. However, work in the area of protein crystallization shows that, due to the highly ordered structure of the lattice, they are far more stable than their amorphous form during delivery in vivo.

Additionally, the dense compaction of a large-sized, crystal structure having uniform morphology allows for much higher drug loading per unit volume with a lowered rate of dissolution. When combined, these characteristics make crystals promising candidates for extended release, carrier-free systems. In known studies, drug-eluting stents, coated in ˜3-5 μm thick crystalline rapamycin for the mitigation of in-stent restenosis in angioplasty, continued to steadily release drug in vitro for up to 75 days and showed good biocompatibility with no inflammatory activity in SD rats. The use of macroscale (micron to millimeter) crystal constructs was recently further expanded upon for long-term controlled release, synthesizing monoliths of the colony stimulating factor 1 receptor (CSFIR) inhibitor GW2580, as well as other compounds, for use in suppressing foreign body and immune rejection responses to implanted biomedical devices, for over 1.3 years in rodents and 6+ months in non-human primates. Therefore, large crystal therapeutics show great potential to act as local reservoirs for extended drug release for a wide variety of afflictions. However, at present, little work has been done on investigating the feasibility of crystalline chemotherapeutic reservoirs for treating tumors. In studies utilizing the optimized crystallization and processing of the multi-tyrosine kinase inhibitor sorafenib, may prove advantageous when developed as an anti-angiogenic therapy or anti-angiogenic composition. Following synthesis, the tunability of crystal size and release kinetics as well as crystalline lattice properties in vitro may be characterized. Efficacy of local crystal sorafenib deposition in a mouse triple negative 4T1 breast cancer (TNBC) model, grown orthotopically in syngeneic BALB/c mice can be tested as well. Uniformly sized batches of 5 different amounts of crystal-formulated sorafenib have been compared to more traditional daily and globally disseminated bolus sorafenib delivery. Lastly, additional tumor tissue-based pharmacodynamic analyses are shown to elucidate drug effects when used in either solubilized or crystallized regimens.

### Crystallization of the Multi-Receptor Tyrosine Kinase Inhibitor Sorafenib

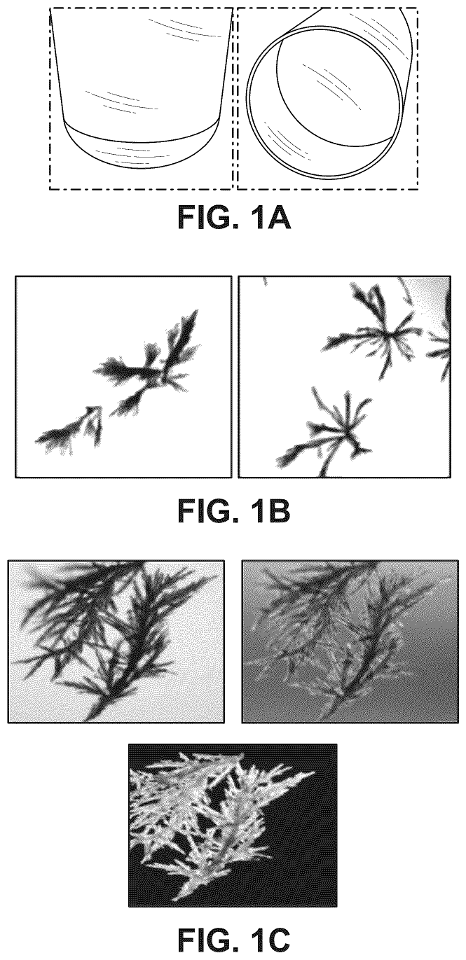

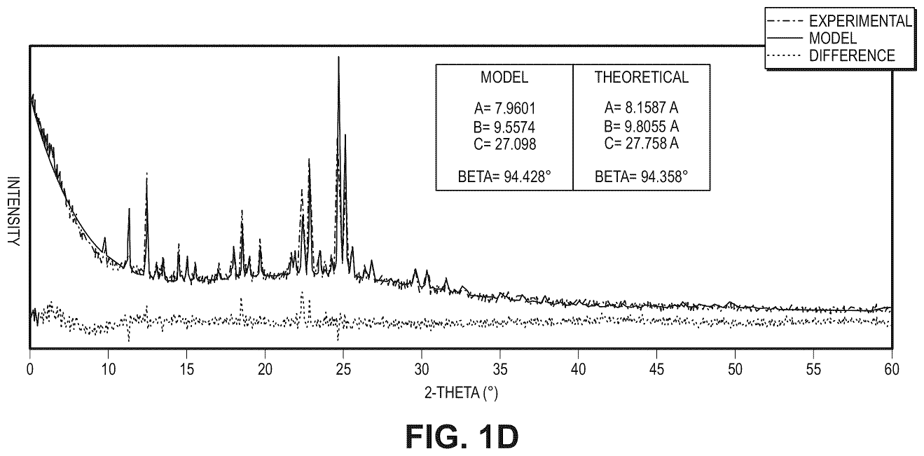

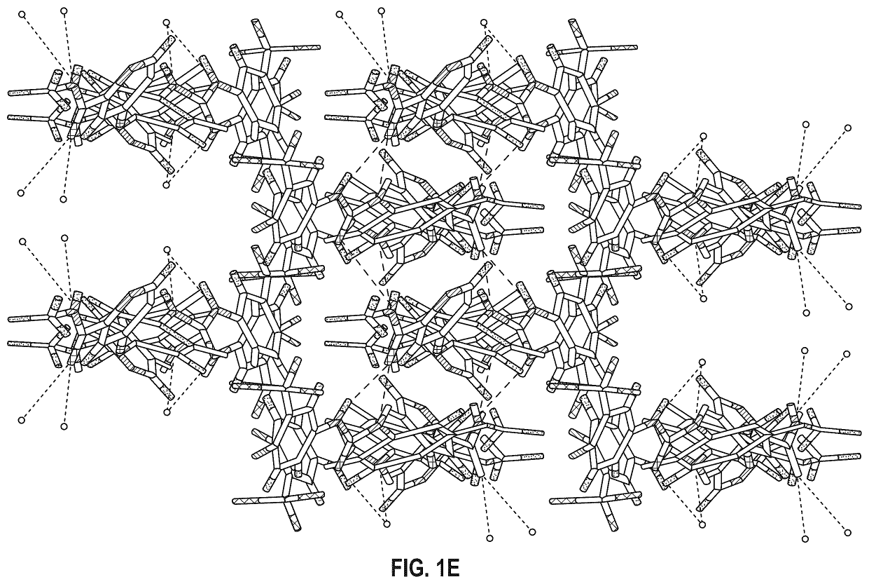

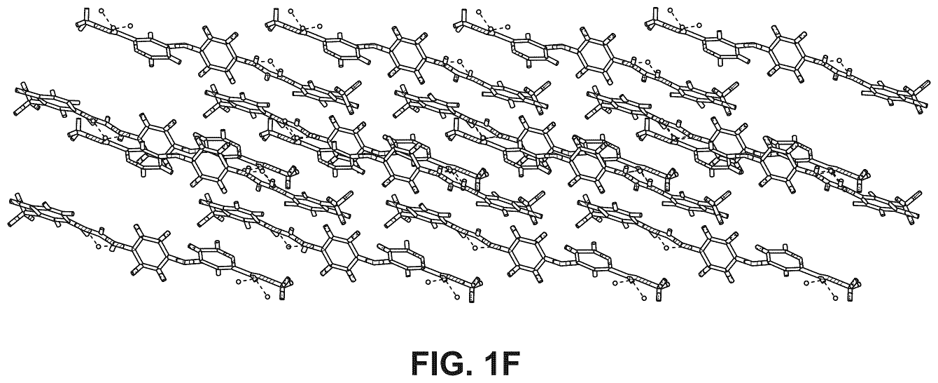

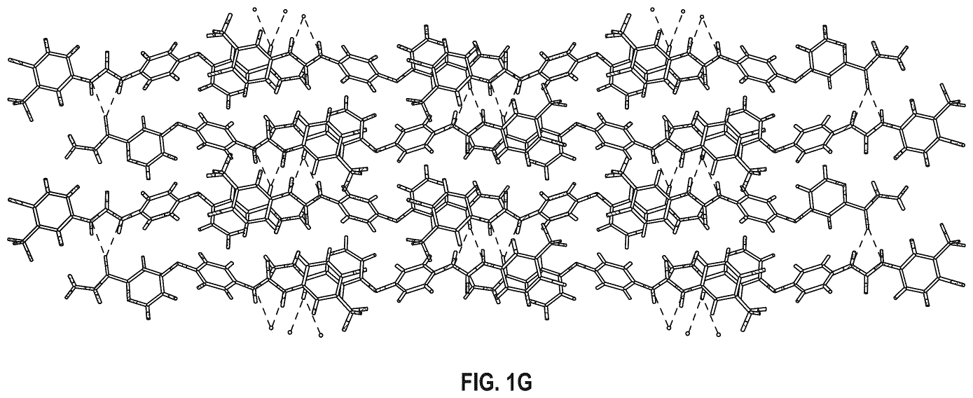

FIGS. 1A-1G depict several views and analyses related to development and characterization of sorafenib crystals, according to an embodiment. FIG. 1A shows two representative depictions of sorafenib crystals forming inside glass vials containing solvent:anti-solvent solution mixtures. FIG. 1B illustrates brightfield microscopy images of sorafenib crystals produced from two different solvent:antisolvent mixture ratios (1:4 and 1:6) of ethyl acetate:hexane at an optical magnification of 4×. FIG. 1C, at left and middle, shows two brightfield images taken of large, branched sorafenib crystal at two different aperture settings; and at right, a phase contrast image taken of the same crystal, at a magnification of 10×. FIG. 1D is a Powder X-Ray Diffraction (PXRD) polymorph analysis of the multireceptor tyrosine kinase inhibitor sorafenib. FIGS. 1E-1G are examples of the 3D unit space packing pattern inside sorafenib crystals as revealed by single crystal X-Ray Diffraction (SXRD), showing views down z, y, and x-planes, respectively. The unit space dimensions are shown in each plane. The foregoing FIGS. 1A-1G reflect representative data and results from in vitro experimental analyses repeated at least 2-3 times.

The solvent/anti-solvent crystallization reaction method used herein is somewhat distinctive in that it does not necessarily have to begin with a supersaturated solution, merely a homogenous, fully dissolved solution. In order to reduce overall solubility, an anti-solvent, or a liquid in which the solute is poorly soluble, is then added to the system, effectively controlling the rate of supersaturation. This method is versatile in its application, as uniform concentration is generally easy to achieve with good mixing, and less equipment is required than cooling crystallization, so it is often used as a secondary step in addition to seeding or cooling to increase yield. As with cooling crystallization, it is important to change the overall concentration of the solution slowly so as to control nucleation and precipitation. It is also important to choose the correct solvent:anti-solvent ratio for the given material, as these can strongly affect yield and morphology. Though this may be less cost effective for batch processes on the industrial scale due to the amount of anti-solvent sometimes required, as well as the need for an additional step to separate the product from the mother liquid, these are less significant considerations at the laboratory scale, making this method advantageous for initial tests on synthesis.

Once a viable crystallization strategy utilizing a solvent:anti-solvent system for the multireceptor tyrosine kinase sorafenib was developed, further work was done to optimize solvent ratios for best yield and morphology, as illustrated in FIGS. 1A-1C. Known small volumes of solvent were added to powdered drug, agitated briefly, then allowed to sit for ten minutes. If dissolution was not achieved, solvent addition was repeated until all material dissolved. Known amounts of antisolvent were added to samples with an anti-solvent-to-solvent ratio of anywhere from 0.5× to 6×, then allowed to sit at room temperature from between 4-24 hours to allow full precipitation, resulting in a controlled crystallization process. As is typical in crystallization processes, reagents and their ratios (two different solvent:anti-solvent mixture ratios (1:4 and 1:6) of ethyl acetate:hexane) did have a noticeable effect on resultant drug morphology as shown in FIGS. 1B-1C. Depending upon the method of crystal formation and growth, a single chemical may be subject to a number of polymorphs, significantly different molecular structural arrangements of the same solid compound. Understanding various polymorphs is important, as each form has its own unique set of thermal, mechanical, and functional properties. Similarly, polymorphs of a single drug may have drastically different characteristics, including solubility in various media, which affects dissolution and bioavailability in the body. Regulatory agencies generally require that a polymorph of a therapeutic be defined as well as the chemical structure. Hence, a key issue in production is being able to control the formation of specific polymorphs. To ensure that only a single crystal form (polymorph) of sorafenib was produced by the described solvent:anti-solvent method, a representative batch of crystals was measured and characterized by powder x-ray diffraction (PXRD), as shown in FIG. 1D and Table 1. Importantly, measured spectra (EXPERIMENTAL) confirmed the presence of only a single drug form, and was nearly identical (DIFFERENCE) to that predicted by a model-based prediction (MODEL) based on theoretical drug parameters. To further characterize the single crystal form, single crystal X-Ray Diffraction (SXRD) was performed to observe the repeating unit space down each axis of the crystalline lattice, as shown in FIGS. 1E-1G. Dimensions of the repeating unit space, demonstrating high density packing of single molecules of sorafenib are shown in each respective plane. Table 1 further lists the 2θ position and intensity of the characteristic diffraction peaks measured for the single crystal polymorph of sorafenib, the crystalline structure as characterized by powder x-ray diffraction (PXRD).

TABLE 1
Powder X-Ray Diffraction (PXRD) data of single
crystal polymorph of sorafenib as produced
by the described solvent: anti-solvent method

| | 2θ | Intensity |
|---|---|---|
| Type | Position | (I) |
| FP | 9.82873 | 593.499 |
| FP | 11.36714 | 811.000 |
| FP | 12.49610 | 1196.988 |
| FP | 13.09351 | 479.000 |
| FP | 13.52342 | 473.455 |
| FP | 14.51222 | 616.412 |
| FP | 15.06210 | 466.518 |
| FP | 15.58330 | 478.244 |
| FP | 17.07999 | 422.824 |
| FP | 18.03658 | 590.437 |
| FP | 18.55550 | 889.849 |
| FP | 19.02613 | 505.354 |
| FP | 19.71383 | 655.529 |
| FP | 21.95166 | 549.868 |
| FP | 22.42198 | 1061.197 |
| FP | 22.87300 | 1285.112 |
| FP | 23.52323 | 563.940 |
| FP | 24.28955 | 542.356 |
| FP | 24.72052 | 2020.233 |
| FP | 25.58707 | 585.924 |
| FP | 26.39713 | 454.650 |
| FP | 26.84406 | 484.000 |
| FP | 29.60360 | 418.034 |
| FP | 30.32969 | 368.217 |
| FP | 31.56384 | 306.129 |
| FP | 36.43163 | 206.232 |
| FP | 37.90511 | 201.391 |
| FP | 40.07659 | 180.040 |

Once optimal formulations were selected, the batch process was scaled up to generate inventory for further characterization and analysis, as well as for use in therapeutic models. Proof of concept formulation exploration was focused on determining a solvent/anti-solvent that would yield crystals while minimizing material drug used, and thus was performed in 4 mL borosilicate vials. Formula optimization, performed in either an 8 mL or 20 mL vial depending on the required total volume for a successful reaction, was focused on optimizing the required ratio of drug to solvent to anti-solvent in order to yield consistent uniform crystals. Once formulation conditions were set, material was synthesized in 500-1000 mL bottles for large batch scale-up, to produce enough material for thorough in vitro testing and in vivo use.

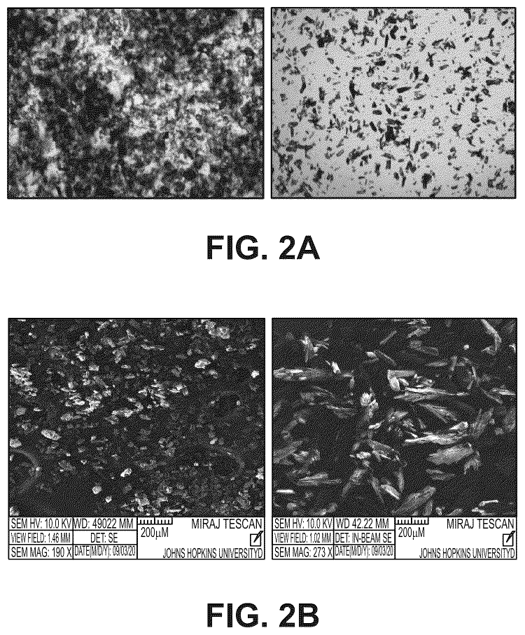

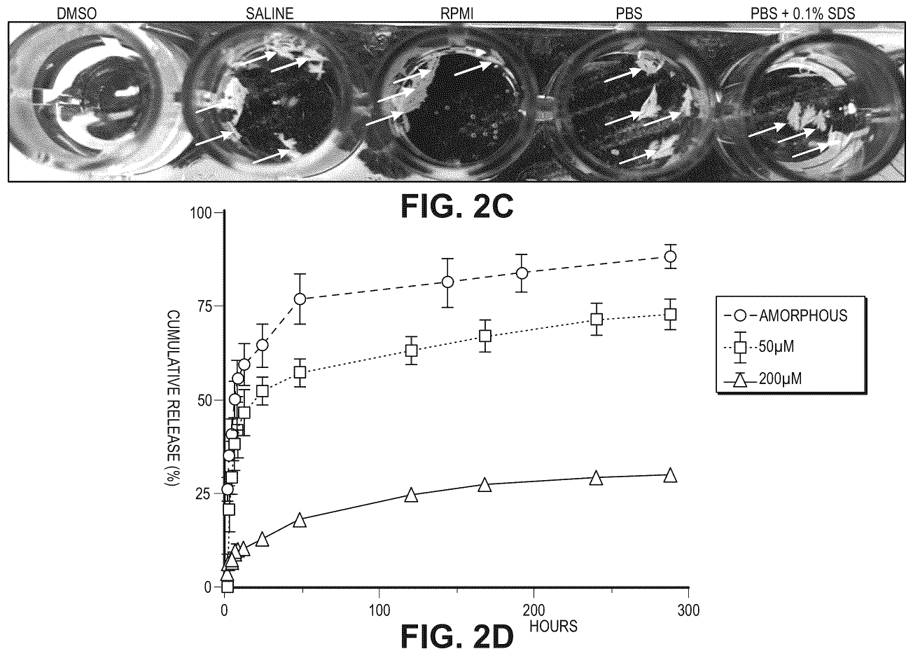

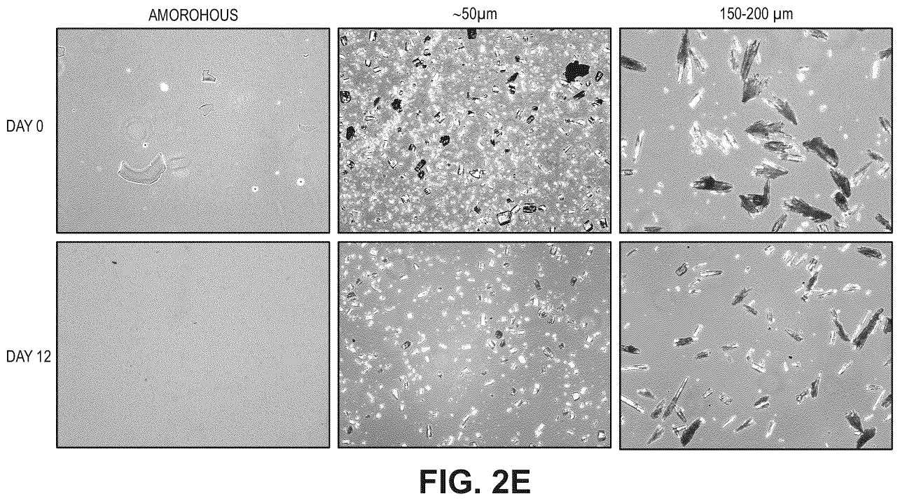

FIGS. 2A-2E depicts various solution stability and tunable release metrics and data related to sorafenib crystals, according to an embodiment. FIG, 2A shows two brightfield microscopy images of two differently sized, ˜50 μm vs. 150-200 μm, respectively, sorafenib crystal preparations, produced by sonicating the original larger, branched crystals, as shown in FIGS. 1B and 1C, at a magnification of 4×. FIG. 2B shows two representative scanning electron microscope (SEM) images of small crystals of ˜50 μm (left) vs. large crystals of ˜150-200 μm (right) of the multi-tyrosine kinase-targeted small molecular inhibitor sorafenib having a scalebar of 200 μm. In certain examples, the multi-tyrosine kinase-targeted small molecular inhibitor sorafenib can be produced with a particle size of from about 1 μm to about 500 μm, or from about 5 μm to about 200 μm, or from about 50 μm to about 150 μm, in accordance with the present disclosure. In exemplary procedures for preparation of crystalline sorafenib, further processing can be employed to reduce or classify the sorafenib into smaller particle sizes, such as but not limited to sonication and filtration through a 10 μm filter. This physical exclusion of larger crystal sizes allows for injection via smaller needles, such as but not limited to, a 5-microliter syringe with the use of a saline carrier. FIG. 2C depicts a photo of sorafenib crystals dissolved in strong organic solvent DMSO as well as saline, RPMI media, 1X PBS, and 1X PBS+0.1% SDS, demonstrating solution stability in the various aqueous solutions, indicating likelihood of long-term stability under in vivo conditions. FIG. 2D is a data plot illustrating in vitro measurement of release under accelerated release conditions (37° C., PBS+0.3% SDS) for amorphous (powder) sorafenib vs. crystalline sorafenib at two different sizes of 50 and 150-200 μm. The fastest release occurred with amorphous powder, while release from crystalline formulations was tuned by varying crystal. The trendlines are shown as a mean value with+/−standard deviation of values. FIG. 1E shows brightfield images showing all sorafenib formulations used for the in vitro release assay, both at the Day 0 start (top) as well as at the end of a full 12-day time course (bottom), at a magnification of 10×. For all in vitro analyses, there was an N=3 technical replicates/group. All data reflects representative data from in vitro experimental analyses repeated at least 3 times.

Typically, in a batch process, additional agitation is provided in the form of a slow mixer to ensure homogeneity throughout the vessel. It is key to keep nucleated crystals in suspension, as crystals that settle to the bottom of a vessel after precipitation are no longer exposed to fresh, saturated solution. Due to the small volumes of the proof of concept and optimization vessels, mixing by fluid flow from pouring was deemed sufficient. In the 500 mL vessel, though the mixing between solvent and anti-solvent from pouring was likely sufficient to achieve an initial homogenous solution, normal molecular movement in the vessel was likely not enough to ensure consistent nucleation and growth across the entire vessel. As a result, effectively two separate layers were seen: one on the bottom of the vessel, smaller crystals precipitated out early, reaching about 300 μm maximum as estimated by observation under brightfield microscopy, while towards the top of the vessel, larger crystals were observed adhering to the sidewall, likely having been exposed to sufficiently saturated solution for longer and growing to as large at 0.5 cm. As the anticipated application of the crystals only required a maximum size closer to 200 μm, the wide size distribution was not as significant in this instance, as milling and shearing by sonication would still be required to decrease overall size.

After crystals visibly stopped precipitating or growing, the vessel was submerged in an ultrasonic water bath for 10-30 seconds to dislodge crystals from the vessel sidewall and break them into more a uniform size distributions, either smaller fines, for example 50 μm, or between 150-200 μm, as measured by brightfield and scanning electron microscopy, shown in FIGS. 2A-2B. Resultant product was then removed from the supernatant mother solution, dried, weighed, and stored for later use. On average, yielded crystal was approximately 40% of the starting powder. Furthermore, methods exist to quantify solubilized material, typically using optics to identify changes in light transmittance, and enabled testing of crystal stability in a range of aqueous solutions. To do so, between 1-2 mg of crystal was placed in a 96-well plate with 300 μL of DMSO, saline, RPMI-1640 media, 1X phosphate buffer solution (PBS), or saline with 0.1% sodium dodecyl sulfate (SDS). With replacing solutions every hour, no noticeable difference was seen in crystal size by brightfield microscopy after 36 hours, except in the case of DMSO control, shown in FIG. 2C. Therefore, to further test tunable in vitro release of differently sized crystals, SDS % in 1X PBS was increased to 0.3%, and a larger amount of drug (2.9 mg) for 3 different formulations-amorphous, 50 μm crystals, or 150-200 μm crystals—was placed into individual wells of a 6-well plate, with 3 mL of accelerated release media added per well. To simulate cumulative drug release over time, all 3 mL supernatant volumes were removed and replaced with completely fresh release media at desired times of 1 hr, 4 hr, 6 hr, 8 hr, 10 hr, 1 day, 2 days, 5 days, 7 days, 10 days and 12 days, and 200 μL aliquots were measured in individual wells of a 96-well plate at 266 nm to determine solubilize sorafenib release, as shown in FIG. 2D. Fastest release occurred with amorphous powder, while release from crystalline formulations was tuned by varying crystal size resulting from the various steps in the crystallization process.

### Antitumor Efficacy of Crystallized Sorafenib Against a Syngeneic 4T1 Breast Cancer Model

Despite advances in cancer research in the past few decades, breast cancer remains the most common form of cancer diagnosed and the second leading cause of cancer-related mortality amongst women, with approximately 1.3 million new cases being diagnosed annually. However, for the majority of these patients, fatal outcomes are not associated with the primary tumor, but with metastatic growth. Early diagnosis of the primary tumor allows for removal by surgery with good outcomes; however, detection commonly takes place after metastasis has already occurred, sometimes in instances in which surgery is not possible. Global chemotherapy can increase the 15-year survival rate by 10%; however, as previously discussed, chemotherapy often has substantial side effects that negatively impact a patient's quality of life. Furthermore, 15-20% of all breast cancer diagnoses are triple-negative breast cancer (TNBC), lack expression of estrogen receptor (ER), progesterone receptor (PR), and epidermal growth factor receptor 2 (EGFR2). Due to the loss of these receptors, typical hormonal-targeted treatments, such as trastuzumab, used to treat breast cancers are unavailable for use. As a result, the five-year survival rate of TNBC drastically decreases once metastasis occurs, 65% for those with regional metastasis, and only 11% for those with distal metastasis.

One of the most commonly used models for pre-clinical trials for breast cancer is the orthotopic implantation of 4T1 mouse TNBC cells into syngeneic immunocompetent BALB/c mice. Of relevance, 4T1 grows rapidly and displays aggressive metastasis, spreading from the mammary fat pad to the lungs, liver, and bone, strongly mimicking the behavior of TNBC in humans. Like human TNBC, 4T1 tumors show irregularly high microvessel density, indicative of its aberrant angiogenesis. Overexpression of VEGF is a common hallmark of cancer, and VEGFR1/2 are expressed more highly in TNBC than any other breast cancer. However, direct inhibition of VEGFR has been shown to have limited capability in preventing tumor recurrence long-term. In general, receptor tyrosine kinases, transmembrane cell-surface receptors that regulate a number of cellular functions including survival and proliferation, can be susceptible to pathway compensation. Therefore, it is possible that a more robust, multi-target RTK inhibitor such as sorafenib may present a more viable treatment option. Furthermore, the use of a successfully crystallized sorafenib, which inhibits a number of angiogenic-related pathways, may prove its utility as well.

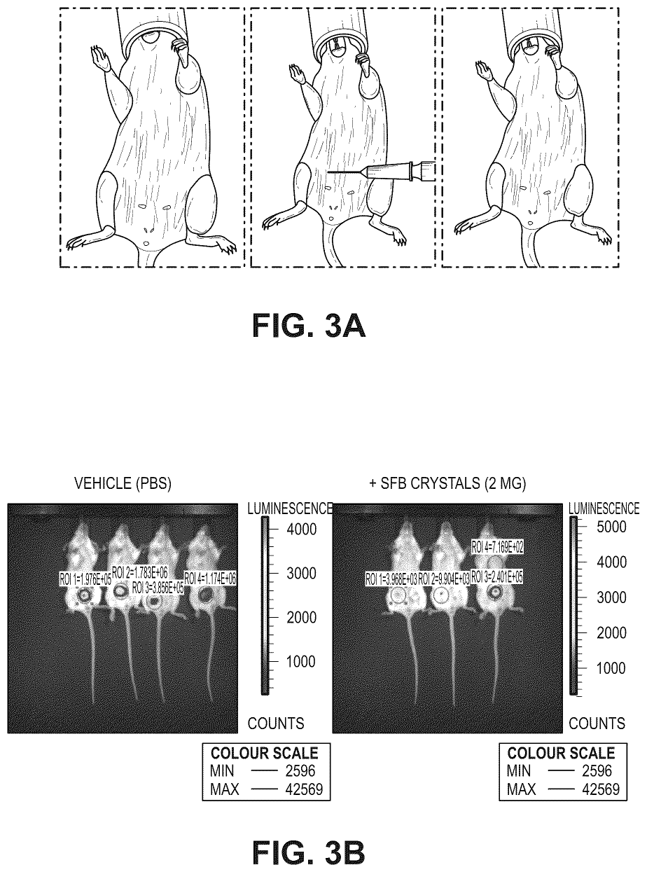

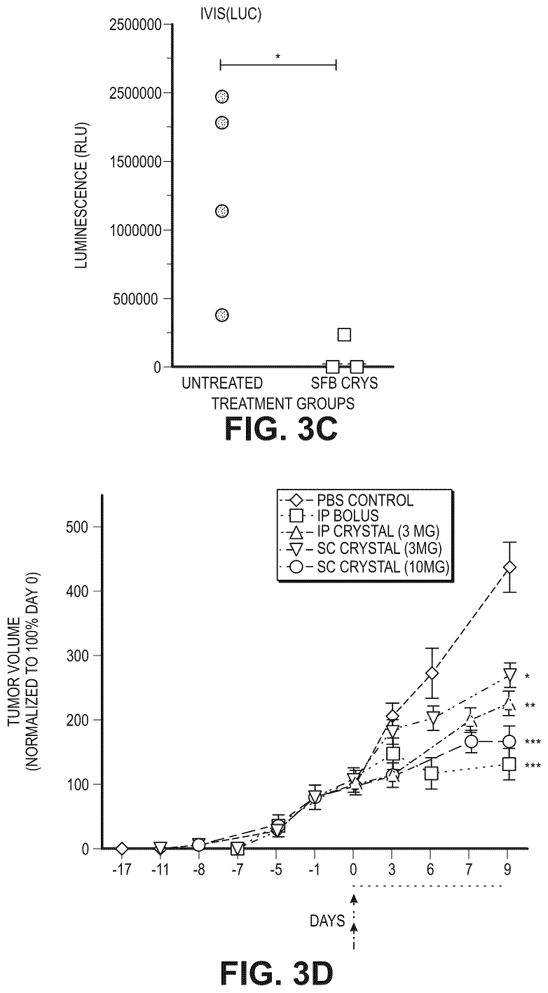

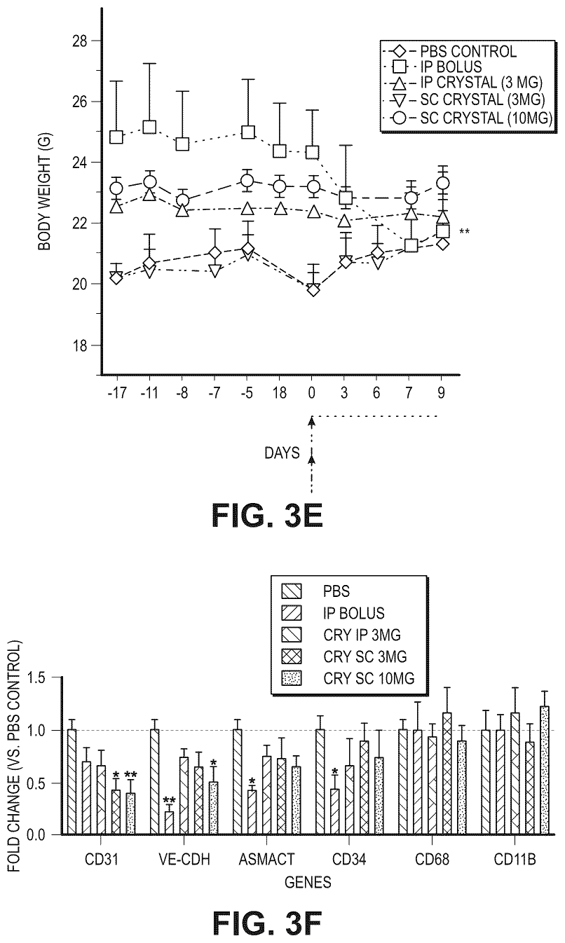

FIGS. 3A-3F are a series of methods and data related to evaluation of anti-tumor effects associated with single delivery of crystalline sorafenib, according to an embodiment. Anti-tumor effects and associated long-term potential of crystal-formulated sorafenib maintained with a single delivery of crystalline sorafenib may be determined using a single injection at Day 0 as compared to daily, solubilized sorafenib injections (25 mg/kg body weight (BW)/day) in a syngeneic model of treating triple negative breast cancer (TNBC): 4T1 tumors grown orthotopically in the mammary fat pad of BALB/c mice. FIG. 3A depicts in vivo testing, for which mice were anesthetized with inhaled isofluorane (left), inoculated with 4T1 (or 4T1-Luc) cancer cells, injected orthotopically into the right inguinal mammary fat pad (middle), with the injection needle subsequently withdrawn (right). FIG. 3B depicts whole body IVIS imaging following luciferin substrate injection to monitor and compare 4T1-luciferase control tumors (left) vs. those co-injected with crystalline sorafenib (right), 20 days post-injection. The 4T1-luciferase is shown where tumor cells are. FIG. 3C illustrates quantification of regions of interest (ROIs) around tumor signatures (minus background), as shown in FIG. 3B. The data shown in FIG. 3C shows individual tumor Relative Luminescence Unit (RLU) measurements. An unpaired T-test, * shows a p<0.05. FIG. 3D is a data plot of growth curves for parent syngeneic 4T1 tumor cells grown in BALB/c mice treated with PBS (PBS CONTROL), intraperitoneal (IP BOLUS) bolus sorafenib (25 mg/kg BW/day), 3 mg IP injected sorafenib crystals (IP CRYSTAL 3 MG), or sorafenib crystals (3 mg: SC CRYSTAL (3 MG); 10 mg: SC CRYSTAL (10 MG)) injected subcutaneously (SC) around developed tumors. Treatment started when tumor volumes reached ˜225-250 mm³ in size. FIG. 3E is a data plot showing body weight curves, in grams (g), for BALB/c mice treated with PBS (PBS CONTROL), intraperitoneal (IP) bolus sorafenib (25 mg/kg BW/day) (IP BOLUS), 3 mg IP injected sorafenib crystals (IP CRYSTAL 3 MG), or sorafenib crystals (3 mg: SC CRYSTAL (3 MG); 10 mg: SC CRYSTAL (10 MG)) injected subcutaneously (SC) around developed tumors. FIG. 3F is a plot exhibiting angiogenesis (endothelial cell marker Cd31, vascular endothelial-cadherin (VE-Cdh), smooth muscle pericyte marker alpha Smooth Muscle actin (aSMact), and stem/progenitor cell marker Cd34) and immune (macrophage Cd68 and myeloid Cd11b) marker expression levels in tumors isolated at the end of this pilot study (day 9) were analyzed by qPCR. Data, analyzed by One-way ANOVA with Tukey multi-comparison exhibits the following: *: p<0.05, **: p<0.01, ***: p<0.0001, versus control tumors (PBS). Bars, mean±SE for n=4-5tumors/group. Experiments were repeated at least twice.

To determine long-term potential of tunable, slow-releasing crystal-formulated sorafenib, efficacy of a single injection of the injectable drug composition was measured in a syngeneic model of triple negative breast cancer (TNBC): 4T1 tumors grown orthotopically in the mammary fat pad of BALB/c mice. For in vivo testing, mice were anesthetized with inhaled isofluorane and inoculated with 4T1, also referred to as 4T1-Luciferase, Luc, cancer cells, injected into the right inguinal mammary fat pad, as shown in FIG. 3A. First, as a proof of concept, whole body IVIS imaging, following luciferin substrate injection, was used to monitor and compare 4T1-luciferase control tumors vs. those co-injected with 2 mg of (150-200 μm) crystalline sorafenib, as depicted in FIG. 3B. Tumor presence was quantified over time up to 20 days post injection in both cases based on measured luminescence in equivalent regions of interest (ROIs, white circles), depicted in FIG. 3C. An unpaired T-test confirmed significant anti-tumor efficacy of sorafenib crystals (p<0.05), as compared to vehicle (PBS)-treated controls. Next, the efficacy of single, injected crystal depots was compared to daily, solubilized sorafenib injections (25 mg/kg body weight (BW)/day).

Since luciferase-expressing 4T1 variants have been shown to display significant limitations, including anti-tumor efficacy artifacts due to the presence of the foreign species-derived xenoantigen (luciferase) 24, parent (non-Luc-expressing) 4T1 tumors were instead used moving forward. Namely, 4T1 tumors were treated with either PBS (PBS CONTROL), intraperitoneal (IP) bolus sorafenib (25 mg/kg BW/day) (IP BOLUS), 3 mg IP injected sorafenib crystals (IP CRYSTAL 3 MG), or sorafenib crystals (3 mg: SC CRYSTAL (3 MG); 10 mg: SC CRYSTAL (10 MG)) injected around developed tumors in the subcutaneous (SC) mammary fatpad. Furthermore, to truly test the utility of crystalline sorafenib in treating such an aggressive TNBC model, treatment was carried out retrospectively and only started once tumor volumes reached ˜225-250 mm³. A positive control liquid-dissolved and daily injected drug treatment group was included, where mice were dosed with a daily bolus of 25 mg sorafenib/kg of mouse BW. Tumor responses to local subcutaneous crystal injections was somewhat limited with tumor delays only, until we increased the amount of injected crystalline drug from 2-3 mg up to 10 mg, upon which tumor stasis more similar to the positive bolus IP drug treatment was achieved, as depicted in FIG. 3D. Furthermore, IP dosing of the crystal, at a 3 mg dose, showed less response than 10 mg SC but more than a comparable 3 mg dose injected SC. This may be supported by our prior observation that in the IP compartment, where there is higher 10 liquid turnover as compared to the SC space, crystalline drugs usually have a faster kinetic of release over time.

However, due to the highly hydrophobic nature of sorafenib, its daily 25 mg/kg bolus dosing regimen required regular used of a solvent solution made up of ˜60% of the strong organic solvent dimethylsulfoxide (DMSO). As a consequence, there was significant body weight loss (˜15-20%) in this treatment group, shown in FIG. 3E, which may have contributed to the slight dip in tumor volumes observed in mice treated with this regimen. In stark contrast, no DMSO was required for delivery of sorafenib crystals, for which instead simply utilized ˜100-200 μl of saline to inject into the desired IP or SC tissue sites. In this instance, it demonstrates that crystal injection groups did not negatively affect bodyweight in any site or dose tested, evidenced by the data in FIG. 3E. Thus, these observations suggest that the toxicity was not related to the drug itself, but the DMSO solution used for injecting pre-dissolved drug.

To determine the anti-angiogenic efficacy of each sorafenib treatment group, qPCR gene expression analysis was performed on retrieved tumors at the conclusion of the study. In particular, analysis for levels of endothelial cell marker Cd31, vascular endothelial-cadherin (VE-Cdh), smooth muscle pericyte marker alpha Smooth Muscle actin (aSMact), and stem/progenitor cell marker Cd34 were conducted, as shown in FIG. 3F. Of note, as compared to daily liquid bolus drug treatment, localized controlled release by crystalline sorafenib more effectively reduced endothelial cell Cd31 expression levels. While knockdown of vascular endothelial-cadherin (VE-Cdh), smooth muscle pericyte marker alpha Smooth Muscle actin (aSMact), and stem/progenitor cell marker Cd34 was not as significant for crystal-formulated sorafenib, this may be a result of daily, bolus sorafenib diffusing everywhere in the body in effect inhibiting cells in other tissue sites/reservoirs as well, perhaps including circulating endothelial progenitor cells. To rule out off-target effects against immune cells in the tumor, screening for macrophage Cd68 and myeloid Cd11b expression levels was conducted, and no significant changes due to any of the treatment groups (bolus or crystal) were found.

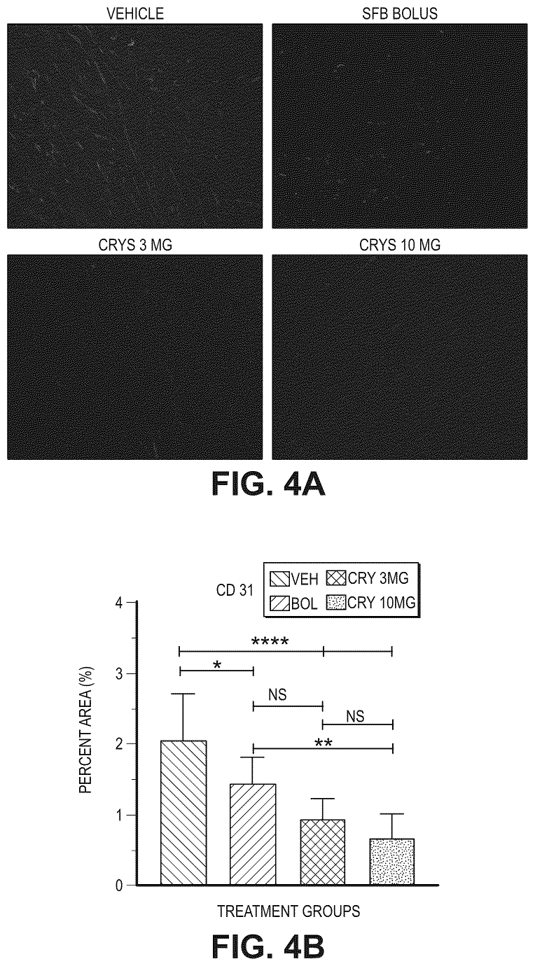

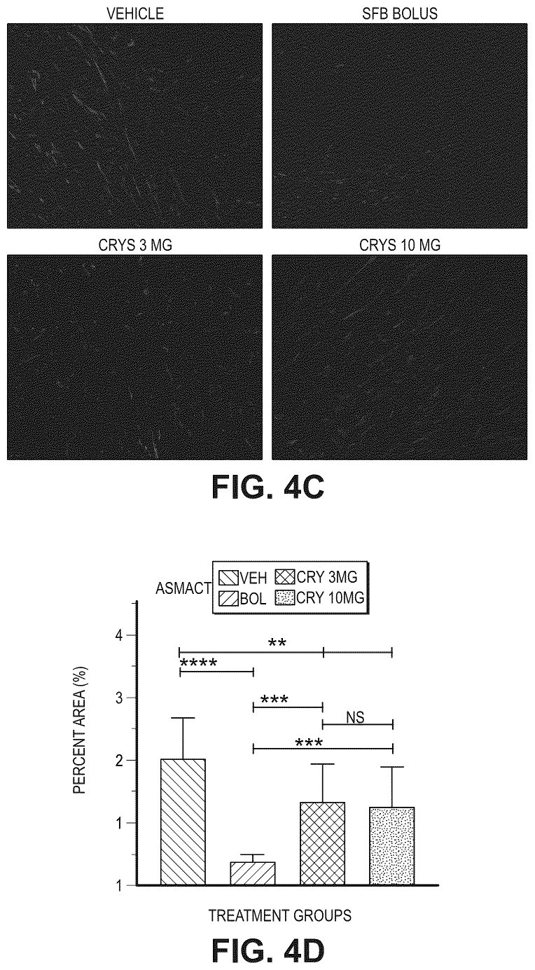

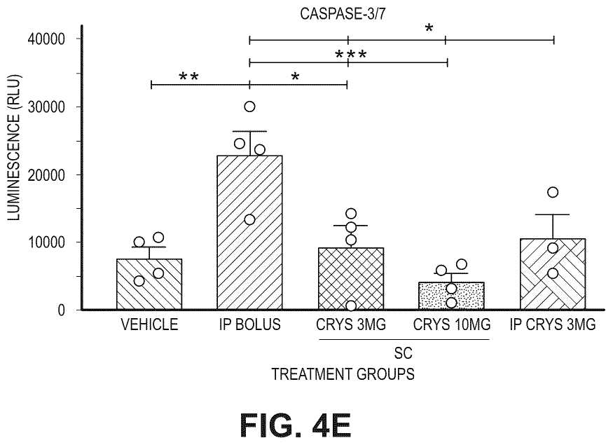

FIGS. 4A-4E depict a series of images and data illustrating anti-angiogenic efficacy of sorafenib crystals, according to an embodiment. Single local injection of slow-release sorafenib crystals maintains effective antiangiogenic efficacy is evidenced by the images and data in FIGS. 4A-4E. FIG. 4A shows fluorescence microscopy images in greyscale, which show significant reduction of tumor endothelial CD31 levels by daily bolus (IP) sorafenib (25 mg/kg/day). However, reduction is greater with a single injection of either 3 mg or 10 mg of locally, subcutaneously (SC) administered sorafenib crystals. Images are at a magnification of 20×. FIG. 4B is a plot of tumor endothelial CD31 presence, expressed in % area, as determined by immunofluorescent staining and ImageJ analysis of the images in FIG. 4A, by analyzing at least 10 fields of view over 3-5 sections per tumor. The experiments were repeated at least twice. FIG. 4C depicts fluorescence microscopy images in greyscale, showing significant reduction of tumor smooth muscle pericyte alpha Smooth Muscle actin (aSMact) levels by daily bolus (IP) sorafenib (25 mg/kg/day). However, reduction is less significant following single injections of either 3 mg or 10 mg of locally, subcutaneously (SC) administered sorafenib crystals. FIG. 4D is a plot of aSMact presence, expressed in % area, as determined by immunofluorescent staining and ImageJ analysis of at least 10 fields of view over 3-5 sections per tumor. FIG. 4E depicts a plot of Caspase-(3/7)-Glo luminescence activity following incubation of tumor lysates, at a level of 10 mg, from each individual tumor in each treatment group as noted. All bars, mean±SE for n=4-5 tumors/group. All data, analyzed by One-way ANOVA with Tukey multi-comparison; *: p<0.05, p<0.01, ***: p<0.001, ****: p<0.0001, ns=no significance; comparisons as noted. Experiments were repeated at least twice.

To confirm the effects seen at the level of RNA, immunofluorescent staining of excised tumor samples that were in parallel fixed with paraformaldehyde and embedded in paraffin was performed. Of note, antibody staining and ImageJ analysis for CD31 as a marker for microvessel density and angiogenesis, confirmed that a single injection event for crystalline drug treatment groups, despite not exhibiting improved anti-tumor efficacy as compared to globally disseminated daily injections of bolus sorafenib, were more effective at reducing endothelial cell presence within treated tumors, as shown in FIGS. 4A-4B. Staining and ImageJ analysis for smooth muscle pericyte marker alpha Smooth Muscle actin (aSMact) exhibited a high amount of co-localization with CD31 in most cases. Furthermore, while decreased in crystalline sorafenib-treated tumors, it was more effectively reduced in mice treated with daily, bolus sorafenib, as evidenced by FIG. 4C-4D. Co-localization of CD31 with aSMact was, as expected, not linked in crystal sorafenib-treated tumors, where CD31 reduction was more extensive than with daily, bolus sorafenib. Without being bound by any particular theory, aSMact, though reduced compared to vehicle treated, control tumors, was higher in crystal treatment groups, it may be attributed to a less effective knockdown of Cd34, perhaps indicative of higher wound healing potential in local crystal-treated tumors. In support of this hypothesis, caspase-3/7 levels in lysates of all tumors collected at the end of the study were measured, and it was found that apoptotic index was significantly increased only in tumors from mice treated with the daily, bolus sorafenib regimen, as evidenced by the data plot of FIG. 4E.

Kinase inhibitor therapies have rapidly been broadened over the past few decades for their perceived ability to target highly specific pathways that become dysregulated due to disease. However, despite great strides, kinases are not fully understood, and as a result, pathway inhibition often leads to unexpected challenges, namely resistance. While receptor inhibition has shown great benefit in cancer treatment, it is not without its drawbacks. Kinase upregulation, pathway compensation, or pathway bypass are all mechanisms of mutation and resistance that frequently occur which are difficult to overcome. At present, sorafenib is clinically approved for the treatment of unresectable hepatocellular carcinoma and advanced renal carcinoma. Though shown to be a potent anti-angiogenic, it tends to exert cytostatic, and not cytotoxic effects. Sorafenib used against breast cancer models has shown varied efficacy. MDA-MB-231, a human derived adenocarcinoma cell line also commonly used to model TNBC, implanted into nude mice was shown to be sensitive to a 30 mg/kg bodyweight daily oral dosing scheme, with 42% tumor reduction after 9 days and significant angiogenic inhibition. However, 20-60 mg/kg dosing of sorafenib against 4T1 in a model similar to those described herein resulted in negligible effects on tumor growth. Further, they observed higher recruitment/polarization of CD206+ F4/80+ M2 macrophages, which are typically associated with tumor associated macrophages and immunosuppression. The qPCR data for immune markers Cd68 and Cd11b as shown in FIG. 3F suggests that there are no significant changes in macrophage/myeloid cell number in response to any of the regimens used in the experiments described herein.

Specificity of treatment is one of the largest gaps in cancer treatment presently. Given that sorafenib targets key cellular RAF kinases, there may be more suitable drugs for inhibiting more specific functions. For example, BCD-115, a small molecule inhibitor of CDK8, is currently in phase I clinical trials and presents an interesting candidate for crystal delivery. Olaparib (marketed as Lynparza) is a clinically approved small molecule poly (ADP-ribose) polymerase (PARP) enzyme inhibitor for the treatment of TNBC. PARP enzymes are involved in cell cycle regulation and DNA repair, which is key for regulating TNBC cells with the breast cancer gene (BRCA) mutation. Either of these may be additionally informative for testing the usefulness of crystalline RTK inhibitors against TNBC.

There are major advantages and disadvantages of both global and local delivery strategies. As previously discussed, global delivery has the distinct drawback that it may affect normal tissues, contributing to systemic effects and overall poor quality of life during chemotherapy. Conversely, in some cases, depending on aggressiveness of metastatic formation, it may be required to clear micro-metastases. Local delivery has the advantage of relegating cytotoxic effects largely only to the affected area. However, it cannot always be assured that circulating tumor cell-type cells shed by the primary tumor will be eliminated before escaping into circulation. Of relevance, the theory of “seed and soil”, proposed by Stephen Paget in 1889, describes the patterns of cancer metastasis, suggesting that tumors shed a heterogenous population of cells that enter the vasculature and lymphatics and migrate to distal tissues which preferentially “seed” accommodating organs, forming secondary tumors. Further studies have shown that these seed cells, or circulating tumor cells (CTCs), colonize distal organs, or even to self-seed an existing tumor, which may explain tumor recurrence even after an original tumor has been excised. This theory was later expanded to also include the aspect that primary cells from the environment secrete chemokines such as osteoponin and stromal-derived factor-1 that may actively play a role in trafficking CTCs, as well as precondition the organ for colonization. Although only approximately 0.01% of CTCs actually reach their preferred soil and successfully form secondary tumors, they may remain dormant and undetected for long periods of time until reactions with their local microenvironment signal reactivation. Upon revival, CTCs immediately begin remodeling, including extracellular matrix remodeling and rapid angiogenesis. Also associated with angiogenesis, circulating endothelial progenitor cells (EPCs) are thought to exhibit regenerative properties in neoplastic disease and exist at abnormally increased levels during metastatic stages of cancer progression. Though their relationship with tumorigenesis is not fully understood, their abundance during tumor progression makes them an ideal surrogate marker for angiogenesis, likely reflecting the high turnover rate of tumor endothelium and the dysregulated nature of vascular growth4. Crystallization strategies should therefore also be further explored for additional efficacy against CTCs and EPCs, and not just primary tumor or tumor-endothelial cells.

In summary, delivery systems that utilize either extended-release principles or are sensitive to changes in the local microenvironment are sought for improved anti-tumor efficacy. Crystals make for a potentially ideal delivery system, as they do not require a carrier and allow delivery of a large amount of drug compacted into a small delivery volume. In addition, their tunability allows for engineering not only new drug forms but unique crystal physicalities that may be utilized to alter pharmacokinetic/dynamic profiles of utilized compounds. Of interest, crystal delivery of pharmaceuticals is a field that, with more groundwork, shows promise in treating disease. It should be noted that other diseases may benefit from treatment with the use of drug delivery systems based on crystals as described herein, such as drug delivery into the eye to slow macular degeneration, for example. Of interest, wet macular degeneration is typified by aberrant formation of blood vessels, through a process called choroidal neovascularization, specifically under the retina and macula in the eye. Of major concern, these newly formed vessels may be leaky discharging fluid and, in some cases, even blood, subsequently leading to pressurization and bulging of the macula. While normally found in a flat orientation, the macula is ultimately lifted out of its normal position, leading to distortion and destruction of a patient's centrally located vision. Furthermore, due to physical distortion and curvature of the eye, straights lines instead take on the appearance of wavy. In such instances, visual impairment may be rapid and painful. Relevancy of anti-angiogenics for the treatment of wet age-related macular degeneration (wet AMD) is clear, but the further benefits of a crystallized depot of an anti-angiogenic that may be locally injected into the vitreous solution of the eye and release slowly for extended durations may lead to significant improvements in preventing abnormal neovascularization, macular degeneration, and vision loss.

### Antitumor Efficacy of Crystallized Sorafenib Against Macular Degeneration

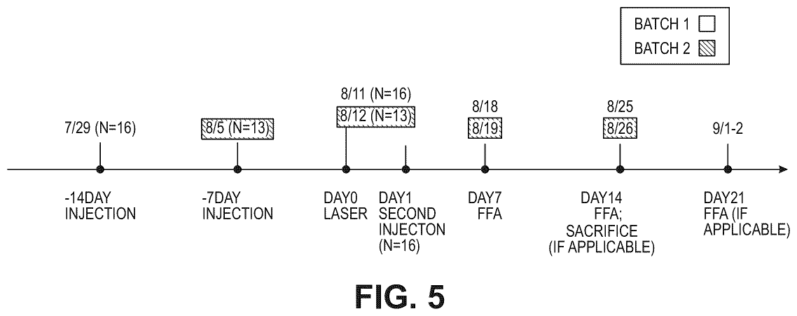

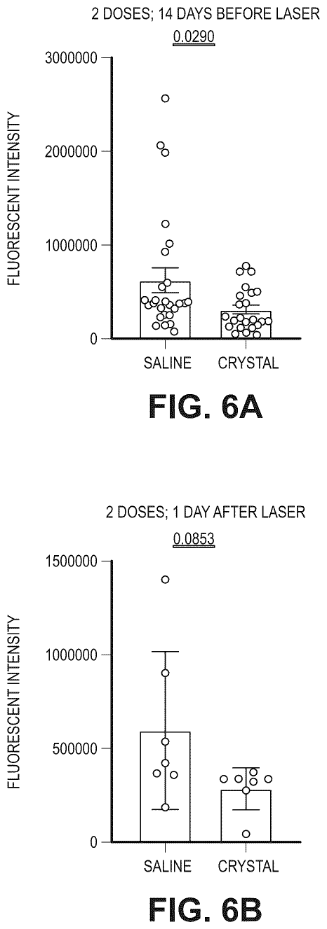

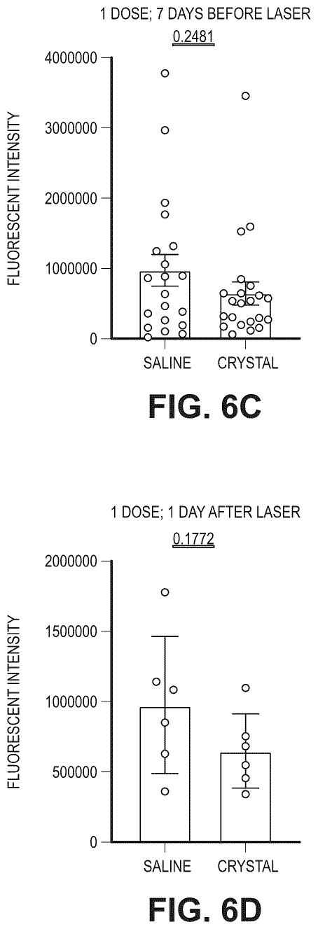

Wet age-related macular degeneration (wet AMD) is a leading cause of blindness in the U.S. and globally, requiring effective treatments, particularly with the use of drugs or treatments that allow sustained management of the disease. Choroidal neovascularization (CNV), or pathologic angiogenesis, is the cause of the visual loss that occurs in wet AMD. Therefore, sorafenib is also a possible treatment for wet AMD, but the effect of the drug when delivered in a liquid form is brief. It has been shown that optimized crystal formulations of sorafenib providing sustained release have the potential to work over a longer period of time. As shown herein, the experiments tested the effect of intravitreal administration of crystal sorafenib vs. vehicle, in saline, of sorafenib in a mouse laser-induced CNV model. FIG. 5 depicts a timeline of an experimental procedure, in accordance with the present disclosure. Crystalline sorafenib, as described herein, was administered to two batches of mice. In the first batch of mice, 16 mice were injected, 14 days before a laser treatment and 1 day after laser treatment. In the second batch of mice, 13 mice were injected 7 days before laser treatment. FIGS. 6A-6D depict charts of fluorescent intensity as measured with the use of fundus fluorescein angiography (FFA), according to the present disclosure. Each chart includes a comparison of a saline dosage as compared to crystal dosage. FIGS. 6A and 6B represent data from the first batch of mice, while FIGS. 6C and 6D represent data from the second batch of mice. Each point represents one lesion in FIG. 6A and 6C and each point represents one eye in FIGS. 6B and 6D. Based on the fluorescent intensity shown in FIGS. 6A-6D, it can be observed that two doses of sorafenib crystal appears to reduce CNV, with statistically significant results, having a p=0.0290 for single lesion analysis. As described herein, preparation of crystalline sorafenib can include further processing to reduce or classify the sorafenib into smaller particle sizes, with, for example, with the use of sonication and filtration through a 10 μm filter. This physical exclusion of larger crystal sizes allows for injection via smaller needles, such as but not limited to, a 5-microliter syringe with the use of a saline carrier. The smaller particle size can provide a crystal small enough to be injected into an eye and other biological feature with restricted physical space. It has been observed in the experiments described in regard to FIGS. 5 and 6A-6D that crystal injected in vitreous fluid of the eye collected 2 weeks after injection have similar cubic shape and are identical in polymorph and crystal form as compared to larger crystalline sorafenib as described herein.

### Materials and Methods

### General Cell Culture and In Vivo Materials

4T1 mouse breast cancer cell line (Cat#ATCC CRL-2539, ATCC), 4T1-Luc2 mouse breast cancer cell line (Cat#ATCC CRL-2539-LUC2, ATCC), RPMI 1640+GlutaMAX cell culture medium (Cat#61870036, ThermoFisher), Fetal bovine serum (Cat#10082147, ThermoFisher), Penicillin-Streptomycin (Cat#15140122, ThermoFisher), Blasticidin S HCl (Cat#A1113903, ThermoFisher), XenoLight RediJect D-Luciferin (Cat#770504, PerkinElmer), and Trypsin-EDTA 0.5% (Cat#15400054, LifeTech/Thermo). Cy3-conjugated anti-mouse alpha smooth muscle actin antibody was purchased from Sigma Aldrich (St. Louis, MO). CD31 antibody was ordered from HistoBioTec (catalog #DIA-310). All the solvents were analytical grade purchased from Sigma 20 Aldrich, USA (as mentioned below). Sodium dodecyl sulfate (SDS) was purchased from Sigma Aldrich, USA.

### 4T1 Cell Culture

4T1 cells were cultured in complete culture media consisting of RPMI 1640+GlutaMAX, 10% FBS, and 1% Penicillin-Streptomycin. During subculture, cells were trypsinized using 0.25% Trypsin-EDTA (LifeTech/Thermo, diluted with DPBS, ThermoFisher). To make frozen cell stocks, cells were stored in complete culture media with 10% dimethylsulfoxide (DMSO) in liquid nitrogen.

### 4T1-Luc2 Cell Culture

4T1-Luc cells were cultured in complete culture media consisting of RPMI 1640+GlutaMAX, 10% FBS, and 8 μg/mL Blasticidin. During subculture, cells were trypsinized using 0.25% Trypsin-EDTA (LifeTech/Thermo, diluted with DPBS, ThermoFisher). To make frozen cell stocks, cells were stored in complete culture media with 10% dimethylsulfoxide in liquid nitrogen.

### Animals

Eight-week-old female BALB/c mice were purchased from Jackson Laboratory. All animal procedures were conducted under IACUC-approved protocols at the Johns Hopkins University.

### Mouse Inoculation

4T1 or 4T1-luc2 cells were trypsinized using 0.25% Trypsin-EDTA, then diluted with the appropriate complete culture media and spun in a centrifuge at 300 g for 5 min. The supernatant was aspirated, and the pellet was re-suspended in serum-free RPMI 1640. The solution was centrifuged again, and the supernatant aspirated. Re-suspension, centrifugation, and aspiration was repeated once more. 5 mL of RPMI-1640 was then added to the pellet. 10 μL of the cell suspension was removed and mixed with 10 μL of Trypan blue stain 0.4% (ThermoFisher). Cells were counted with a Countess automated cell counter. Cell solutions were centrifuged one more time, the supernatant aspirated, and the pellet resuspended in the amount of RPMI-1640 required to achieve a concentration of 5×105 cells/200 μL of media. Tumor cells were then administered orthotopically in naive (and anesthetized) BALB/c mice into the right inguinal mammary fat pad 21 with 200 μL aliquots of the cell suspension, using an insulin syringe. Tumors were measured externally using a digital caliper, and volume was calculated (!″* (length * height) *−length * height). Except in the 4T1-luc2 trial, treatment began once tumors reached an average volume of 220-250 mm3. Experimental end point was deemed when solid tumors reached a volume of no more than 2000 mm3. Tumors were extracted and harvested, half stored in 4% paraformaldehyde diluted from 32% (Cat#50980495, Electron Microscopy Services) with DI H2O, and half frozen in liquid nitrogen, then stored at −80° C.

### 4T1-Luc2 Bioluminescent Tumor Monitoring (IVIS)

4T1-Luc2 tumor-injected mice were dosed with RediJect D-Luciferin K+ salt 5 minutes prior to examination. Bioluminescent imaging was carried out using IVIS Spectrum in vivo imaging system with a luminescent exposure of 0.5-1 second and an aperture F-stop of 1. Equivalent regions of interest (ROIs) were used for quantification of tumor groups. Representative responses are shown in displayed images, along with quantification bar graphs (data: mean+/−SE) with background being subtracted.

### Drug Formulation Materials

Sorafenib (Cat#S-8599, LC Labs), Dimethyl sulfoxide (Cat#D8418-250ML, Sigma), Ethyl Acetate, anhyd. (Cat#270989-2L, Sigma), n-Hexane (Cat#1043742500, Sigma), 4 mL borosilicate glass vials (Cat#14 955 327, Fisher Sci), 8 mL borosilicate glass vial (Cat#03 340 60B, Fisher Sci), 20 mL borosilicate glass vials (Cat#03 340 121, Fisher Sci), 500 mL borosilicate media bottle (Cat#10754-818, VWR), and 500 mL Pyrex wide-mouth storage bottle (Cat#13 700 403, Fisher Sci).

### Methods Optimization and Results for Achieving Crystals of Different Chemotherapeutics

In order to initially assess processability, a testing matrix was developed with each drug matched with available common solvents in ratios of 1 mg drug per 1 mL of solvent. Samples were generated by measuring the drug in a 4 mL borosilicate vial, adding solvent on top, agitating in an ultrasonic water bath for 1 minute to break any agglomerated clumps, and allowing to rest for 1 hour. Any samples that did not dissolve at room temperature were placed on a hot plate at 90° C. for an extra hour. Any combinations that were considered miscible (no visible powder, minimal iridescence in the solution) were then selected to attempt precipitation. Results of the miscibility test are summarized in Appendix A: Tables S1-S2. Solubilized samples were subjected to 3 mL of the antisolvent. Anti-solvent was slowly added dropwise to room temperature solutions, then allowed to precipitate for between 4 to 24 hours.

### Amorphous Drug Preparation

Amorphous material was prepared by first dissolving drug in DMSO (in a minimum volume). Then, into a glass vial on a hot plate (40-50° C.) continuously flushed with N₂, saturated drug solution (drug+minimum volume of solvent) was added in a droplet manner. By first contact between the drug solution and hot glass the solvent evaporates resulting in amorphous drug, which was collected and used for in vitro release studies.

### Crystal Sorafenib Treatment

Once tumors had reached an average volume of ˜220-250 mm³, mice were administered either subcutaneously or intraperitoneally with 3 or 10 mg of crystalline sorafenib, diameter ˜150-200 μm, suspended in 200 μL of sterile saline using an 18 g needle. As a control, mice (n=4) were dosed with sorafenib dissolved at a concentration of 4 mg/mL in a solution of 65% DMSO (remainder DPBS). Mice were dosed either subcutaneously or intraperitoneally with a bolus of 25 mg drug per kg of bodyweight (BW), daily. As a secondary control, mice were dosed with the 65% DMSO/PBS vehicle to monitor changes in body score based on the vehicle alone.

RNA Extraction and qPCR

1 mL of TRIzol reagent (Cat#15596018, ThermoFisher) was added to 100 mg samples of tumor tissue and broken down with a power homogenizer (Polytron/VWR). Homogenized samples were incubated at room temperature for 5 min for complete nucleoprotein dissociation, then 0.2 ml of chloroform (Sigma) was added to each sample. Samples were shaken vigorously for 15 seconds and incubated at room temperature for 3 min before centrifuging at 12,000 g for 15 min at 4° C. The uppermost supernatant layer was pipetted into a 1.7 mL Eppendorf tube, with the remaining interphase and organic layer discarded. 0.5 mL of isopropanol (Sigma) was added to each sample, and then samples were incubated for 10 min. Samples were centrifuged at 12,000 g for 10 min at 4° C. Resultant supernatant liquid was siphoned off, leaving only the RNA pellet. Pellets were 23 washed with 1 mL of 75% ethanol (100% ethanol, Sigma, diluted with DEPC-H2O, Invitrogen), then samples were centrifuged at 7,500 g for 5 min at 4° C. Supernatant was again discarded, and sample pellets were allowed to dry in air for 5-10 min. Depending on size of resultant pellet, pellets were resuspended in 50-100 μL, and nucleic acid concentration was measured using a NanoDrop 2000 spectrophotometer (ThermoFisher). Reverse transcription (RT) of RNA was performed by first measuring nucleic acid concentrations for each sample. 1 μg of RNA was added into separate 0.2 mL PCR tubes (Cat#14-230-225, Fisherbrand). DEPC-water was used to equilibrate each sample volume to 12.2 μl, and then 2 μl of 10× RT buffer, 0.8 μl of dNTPs, 1 μl of DNAse, and 1 μl of RNAse inhibitor was added to each sample (High-Capacity RT Kit, Invitrogen). A DNAse cycle was run to remove any potential contaminating genomic DNA. DNAse-free samples were topped with 2 μl of random primers and 1 μl of reverse transcriptase enzyme, and then placed back onto a ThermoCycler (Applied Biosystems) to reverse transcribe RNA into cDNA. Resultant cDNA was then subjected to expansion and analysis by qPCR using the Power SYBR Green protocol (Applied Biosystems). The following primers (forward and reverse, respectively) were used for CD31: 5′-CCTCAGTCGGCAGACAAGATG-3′ and 5′-GCATAGAGCACCAGCGTGAGT-3′; for VEcadh: 5′-GTGGCCAAAGACCCTGACAA-3′ and 5′-TCACTGGTCTTGCGGATGGA-3′; for aSMact: 5′-CGCTTCCGCTGCCCAGAGACT-3′ and 5′-TATAGGTGGTTTCGTGGATGCCCGCT-3′; for CD34: 5′-ATGCTTACACATCATCTTCTGCTCC-3′ and 5′-CCTCACTTCTCGGATTCCAGAG-3′; for CD68: 5′-GCCCGAGTACAGTCTACCTGG-3′ and 5′-AGAGATGAATTCTGCGCCAT-3′; for CD11b: 5′-CCAAGAGAATGCAAAAGGCTTT-3′ and 5′-GGGGGGCTGCAACAACCACA-3′; and for housekeeping gene bact: 5′-GCTTCTTTGCAGCTCCTTCGTT-3′ and 5′-CGGAGCCGTTGTCGACGACC-3′.

### SXRD and PXRD

SXRD—Diffraction data (φ- and ω-scans) were collected at 100K on a Bruker D8 Focus diffractometer with a LynxEye detector using Cu Kα radiation. All non-hydrogen atoms were refined anisotropically. All carbon-bound hydrogen atoms were placed in geometrically calculated positions and refined using a riding model. Coordinates for hydrogen atoms bound to nitrogen or oxygen were taken from the Fourier difference.

PXRD—Phase purity was checked with Powder X-ray diffraction (PXRD) data collected at room temperature. This was done using Bruker D8 Focus diffractometer with a LynxEye detector using Cu Kα radiation. Rietveld refinements on the PXRD data were done through TOPAS 4.2 (Bruker).

SEM—Crystal size, morphology, and topography were studied with SEM. Samples were placed on a conductive carbon paper and were coated with carbon to a thickness of about 10 nm using a sputtering machine (Polarone E5100). After, samples were imaged using scanning electron microscopy (FEI E-SEM Quanta 2000) at voltages of 2-15 kV. 10 random measurements were taken per image for each studied preparation.

### In Vitro Release (Crystalline or Amorphous)

Release studies were carried out under accelerated conditions in PBS with 0.3% w/v SDS in 1X PBS. All of the in vitro release studies were carried out under sink conditions with 2.9 mg drug placed into individual wells. 3 mL PBS+0.3% SDS were added per well, and the plate was gently shaken prior to placement in a 37° C. incubator to mimic physiological conditions. Sampling was carried out by total replacement of release medium with fresh medium. Sampling time points were 1 hr, 4 hr, 6 hr, 8 hr, 10 hr, 1 day, 2 days, 5 days, 7 days, 10 days and 12 days. At each timepoint, images were first taken, the plate was then tilted to let crystals fall to the bottom, supernatant was pipetted off and stored appropriately. 200 μL of each sample supernatant was taken for subsequent measurement at 266 nm in a UV-compliant 96-well plate. Drug concentration in samples was measured using a UV-calibrated Tecan infinite M200 PRO. Calibration curves were prepared in concentration ranges of 0.05-10 μg/ml. Using prepared calibration curves, drug concentrations in different release samples were calculated. OD readings were averaged together (+/−SD) for duplicate technical replicates per condition per timepoint (with empty well background OD subtracted in all cases). Drug release displayed shown as cumulative (additive) release over time (as % total starting amount).

### Imaging Drug Formulations (New and Over Time)

For brightfield or phase contrast imaging of formulated drug initially or over time (e.g., in vitro release) samples were imaged using an EVOS Stereoscopic microscope.

### Immunofluorescence Microscopy

Immunofluorescence imaging was used to determine anti-angiogenic drug efficacy. Tumor tissues were retrieved from mice and fixed overnight using 4% paraformaldehyde at 4° C. After fixation, retrieved tissue samples were moved to 70% ethanol. Samples were then processed for paraffin embedding, sectioning and staining. For staining, de-paraffinized samples were subjected to antigen retrieval for 45 min. in a rice steamer using Biogenix Citra pH 6 buffer (Cat#HK086-9K), rinsed with 1X PBS 3 times (5 min each), permeabilized for 5 min on ice using a 1% Triton X100 solution (in 1X PBS), rinsed again 3 times with PBS, and subsequently blocked for 1 hour using a Dako blocking solution (Cat#S3022). Next, tissues were incubated for 1 hour in an immunostaining cocktail solution consisting of NucBlue (Thermo), specific marker probes (1:200 dilution) anti-CD31 (Cat#DIA-310, HistoBioTec) and anti-alphaSmoothMuscle actin-Cy3 (Sigma) in Dako staining buffer. For CD31, a goat anti-rat alexa-fluor647 secondary antibody was also used (1:400). After staining, samples were rinsed again with 1X PBS three times prior to slides being cover slipped with Pro-long anti-fade Gold (Thermo). Fluorescent images were acquired with an ApoTome-based AxioObserver.Z2 system (Carl Zeiss Microscopy, Jena Germany) equipped with 5, 10, and 20× objectives. For stain quantification, images were converted into 8-bit image files, a background threshold was determined using NIH ImageJ, and then all images were processed using an NIH ImageJ macro to quantify the area of positive stain. Data shown are group mean±SEM with statistical analysis as outlined below.

### Caspase-Glo

Caspase activity was determined using the Caspase-Glo 3/7 assay kit and the manufacturer's protocol, with the following modifications. Protein samples (prepared by sonication probe in NP40 lysis buffer (Cat#FNN0021, Thermo)+Halt protease inhibitor cocktail (Cat#78425, Thermo)) were prepared in a 96-well plate following BCA assay (Cat#23225, Pierce/Thermo) protein quantification (10 mg in 50 μl) were incubated with 50 μl of reconstituted caspase 3/7 substrate. After a brief shaking, the plate was incubated at room temperature for 1 hr and luciferase activity was determined using a BioTek Synergy 2 plate reader (Biotek; Winooski, VT).

### Statistical Analysis

Data are expressed as mean±SEM, and N=3-5 mice per time point and per treatment group, as indicated. All animals were included in analyses except in instances of unforeseen sickness or morbidity. Animal cohorts where randomly selected. Investigators where not blind to performed experiments. For qPCR, data were analyzed for statistical significance by one-way ANOVA with Tukey multiple comparison correction, unless indicated otherwise, as implemented in GraphPad Prism 9; *: p<0.05, **: p<0.001, and ***: p<0.0001.

While the present teachings have been illustrated with respect to one or more implementations, alterations and/or modifications may be made to the illustrated examples without departing from the spirit and scope of the appended claims. For example, it may be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or embodiments of the present teachings. It may be appreciated that structural objects and/or processing stages may be added, or existing structural objects and/or processing stages may be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items may be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “conformal” describes a coating material in which angles of the underlying material are preserved by the conformal material. The term “about” indicates that the value listed may be somewhat altered, as long as the alteration does not result in nonconformance of the process or structure to the illustrated embodiment. The terms “couple,” “coupled,” “connect,” “connection,” “connected,” “in connection with,” and “connecting” refer to “in direct connection with” or “in connection with via one or more intermediate elements or members.” Finally, the terms “exemplary” or “illustrative” indicate the description is used as an example, rather than implying that it is an ideal. Other embodiments of the present teachings may be apparent to those skilled in the art from consideration of the specification and practice of the disclosure herein. It is intended that the specification and examples be considered as exemplary only, with a true scope and spirit of the present teachings being indicated by the following claims.

What is claimed is:

1. An anti-angiogenic composition, comprising:
a multi-tyrosine kinase inhibitor comprising a compound of structural formula 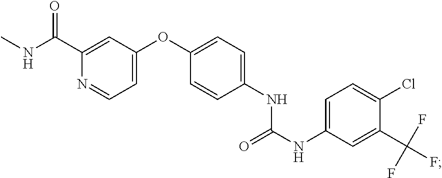 [SMILES: CNC(=O)c1cc(Oc2ccc(NC(=O)Nc3ccc(Cl)c(C(F)(F)F)c3)cc2)ccn1]
and
wherein the tyrosine kinase inhibitor has a crystalline structure comprising a single crystal polymorph.

2. The anti-angiogenic composition of claim 1, wherein the tyrosine kinase inhibitor, as characterized by powder x-ray diffraction, has diffraction peaks at 2θ of 11.37°, 12.50°, 18.04°, 22.42°, 22.88°, and 24.72°.

3. The anti-angiogenic composition of claim 1, wherein the tyrosine kinase inhibitor has a particle size of from about 5 to about 300 microns.

4. The anti-angiogenic composition of claim 1, wherein the tyrosine kinase inhibitor has a particle size of from about 150 to about 200 microns.

5. The anti-angiogenic composition of claim 1, wherein the tyrosine kinase inhibitor is crystallized using a solvent/anti-solvent crystallization reaction.

6. The anti-angiogenic composition of claim 5, wherein the solvent is ethyl acetate.

7. The anti-angiogenic composition of claim 5, wherein the anti-solvent is hexane.

8. The anti-angiogenic composition of claim 1, wherein the tyrosine kinase inhibitor is sonicated.

9. The anti-angiogenic composition of claim 1, wherein the tyrosine kinase inhibitor is injectable.

10. The anti-angiogenic composition of claim 1, wherein the tyrosine kinase inhibitor comprises no carrier.

11. The anti-angiogenic composition of claim 1, wherein the tyrosine kinase inhibitor is hydrophobic.

12. The multi-tyrosine kinase inhibitor composition of claim 1, wherein the tyrosine kinase inhibitor has an anti-angiogenic efficacy as indicated by a reduction in endothelial cell marker Cd31, vascular endothelial-cadherin (VE-Cdh), smooth muscle pericyte marker alpha Smooth Muscle actin (aSMact), or a combination thereof, as compared to a control composition.

13. A multi-tyrosine kinase inhibitor composition, comprising:
a crystalline sorafenib; and
wherein the crystalline sorafenib, as characterized by powder x-ray diffraction, has diffraction peaks at 2θ of 11.37°, 12.50°, 18.04°, 22.42°, 22.88°, and 24.72°.

14. The multi-tyrosine kinase inhibitor composition of claim 13, wherein the crystalline sorafenib has a particle size of from about 5 to about 300 microns.

15. The multi-tyrosine kinase inhibitor composition of claim 13, wherein the crystalline sorafenib has a particle size of from about 150 to about 200 microns.

16. The multi-tyrosine kinase inhibitor composition of claim 13, wherein the crystalline sorafenib is injectable.

17. The multi-tyrosine kinase inhibitor composition of claim 13, wherein the multi-tyrosine kinase inhibitor composition comprises no carrier.

18. The multi-tyrosine kinase inhibitor composition of claim 13, wherein the multi-tyrosine kinase inhibitor composition is hydrophobic.

19. The multi-tyrosine kinase inhibitor composition of claim 13, wherein the multi-tyrosine kinase inhibitor composition is crystallized using a solvent/anti-solvent crystallization reaction, wherein the solvent is ethyl acetate and the anti-solvent is hexane.

20. An injectable drug composition, comprising:
a multi-tyrosine kinase inhibitor comprising a compound of structural formula 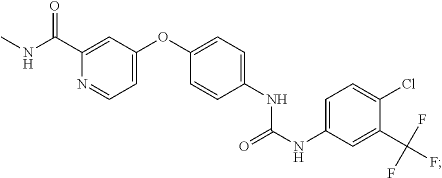 [SMILES: CNC(=O)c1cc(Oc2ccc(NC(=O)Nc3ccc(Cl)c(C(F)(F)F)c3)cc2)ccn1]
wherein:
the multi-tyrosine kinase inhibitor has a crystalline structure comprising a single crystal polymorph;
the multi-tyrosine kinase inhibitor, as characterized by powder x-ray diffraction, has diffraction peaks at 2θ of 11.37°, 12.50°, 18.04°, 22.42°, 22.88°, and 24.72°; and
the multi-tyrosine kinase inhibitor has a particle size of from about 5 to about 200 microns.
